(19) United States
(12) Patent Application Publication
(10) Pub. No.: US 2024/0358908 A1
(43) Pub. Date: Oct. 31, 2024

(54) ANTI-VACUUM SURGE (AVS) WITH IMPROVED FOLLOWABILITY

(71) Applicant: JOHNSON & JOHNSON SURGICAL VISION, INC., Irvine, CA (US)

(72) Inventors: Vadim Gliner, Haifa (IL); Eran Haas, Haifa (IL); Assaf Govari, Givat Nili (IL); Adam Walter Toner, Ladera Ranch, CA (US)

(21) Appl. No.: 18/140,234

(22) Filed: Apr. 27, 2023

(51) Int. Cl. A61M 1/00; A61F 9/007

(52) U.S. Cl. CPC A61M 1/742; A61F 9/00745; A61M 1/73; A61M 1/75; A61M 2039/226; A61M 2205/3341; A61M 2205/505

(57) ABSTRACT

A system includes an anti-vacuum surge (AVS) mechanism and a processor. The AVS mechanism is configured to suppress a vacuum surge during the phacoemulsification procedure. The processor is configured to (i) during the phacoemulsification procedure, receive pressure readings from one or more sensors, (ii) using the pressure readings, estimate an occurrence of a vacuum surge, and (iii) upon estimating the occurrence of the vacuum surge, activate the AVS mechanism in a predefined pulsed manner to suppress the vacuum surge.

### FIELD OF THE DISCLOSURE

The present disclosure relates generally to phacoemulsification apparatuses and probes, and particularly to systems and methods for control of intraocular pressure (IOP).

### BACKGROUND OF THE DISCLOSURE

A cataract is a clouding and hardening of the eye's natural lens, a structure which is positioned behind the cornea, iris and pupil. The lens is mostly made up of water and protein and as people age these proteins change and may begin to clump together obscuring portions of the lens. To correct this, a physician may recommend phacoemulsification cataract surgery. In the procedure, the surgeon makes a small incision in the sclera or cornea of the eye. Then a portion of the anterior surface of the lens capsule is removed to gain access to the cataract. The surgeon then uses a phacoemulsification probe, which has an ultrasonic handpiece with a needle. The tip of the needle vibrates at ultrasonic frequency to sculpt and emulsify the cataract while a pump aspirates particles and fluid from the eye through the tip. Aspirated fluids are replaced with irrigation of a balanced salt solution to maintain the anterior chamber of the eye. After removing the cataract with phacoemulsification, the softer outer lens cortex is removed with suction. An intraocular lens (IOL) is then introduced into the empty lens capsule restoring the patient's vision.

The present disclosure will be more fully understood from the following detailed description of the examples thereof, taken together with the drawings in which:

### BRIEF DESCRIPTION OF THE DRAWINGS

FIG. 1 is a schematic, pictorial view, along with an orthographic side view, of a phacoemulsification apparatus, in accordance with an example of the present disclosure;

FIGS. 2A and 2B are graphs that schematically show different levels of vacuum surge suppression with resulting respective followability levels, in accordance with examples of the present disclosure;

FIG. 3 is a schematic, pictorial illustration of different waveforms applied to an anti-vacuum surge (AVS) module to suppress a vacuum surge, in accordance with examples of the present disclosure; and

FIG. 4 is a flow chart schematically illustrating a method to suppress a vacuum surge while maintaining followability, in accordance with an example of the present disclosure.

### DETAILED DESCRIPTION OF EXAMPLES

### Overview

During phacoemulsification of an eye lens, if an emulsified lens particle fully blocks the inlet of the aspiration needle tip, a high vacuum occurs in the aspiration line. When the aspiration line becomes unblocked (e.g., by the particle being subsequently sucked into the line), the high vacuum in the line leads to the eye experiencing a surge in aspiration suction power (also called hereinafter a “vacuum surge”) with potentially traumatic consequences to the eye as the IOP abruptly falls. For example, exposing the eye to suction power leading to a too low IOP (e.g., below −20 mmHg) may cause the eye chamber to collapse.

A recent solution to the problem of automatically detecting and mitigating vacuum surges is described in U.S. patent application Ser. No. 17/130,409, filed on Dec. 22, 2020, and titled, “A module for Aspiration and Irrigation Control,” whose disclosure is incorporated herein by reference. The application discloses an anti-vacuum surge (AVS) mechanism (e.g., an AVS module), coupled with the phacoemulsification probe, which prevents a sudden vacuum increase being transferred to the eye in case of occlusion break (i.e., prevents vacuum surge). For example, the module can mitigate the vacuum surge by closing off a connection from the aspiration line to the eye at the distal side of the module as soon as a vacuum surge is detected.

To avoid vacuum surge inside the eye, the AVS module very quickly (e.g., within a few tens of milliseconds) cuts off the aspiration line. However, blocking aspiration indefinitely by closing the AVS valve compromises “followability.” Followability is defined as the ability to attract cataract pieces to the phacoemulsification tip. Sufficient followability is important for efficient and safe phacoemulsification. An overly reactive system (or user) regarding vacuum surges as the tip is placed or moved within the capsular bag causes the system to fully interrupt suction (e.g., using the AVS mechanism) in cases of IOP drop, and such overreaction to AVS operation degrades followability.

As the physician must still avoid the aforementioned traumatic consequences to the eye due to vacuum surges, such a restriction to followability may cause the physician to perform the phacoemulsification process too slowly, thereby leading to practical ineffectiveness.

Examples of the present disclosure that are described hereinafter provide methods and systems to allow a physician performing phacoemulsification to operate the AVS in a modulated manner. Instead of cutting off the aspiration line completely, the disclosed technique pulses the AVS valve “on” and “off” at a high frequency (e.g., of about 1 KHz). The pulsing may both avoid reduction in followability and protect the eye against a vacuum surge.

In one example, the AVS is pulsed at increasing intervals (e.g., at a decreasing duty cycle) so that the breaking of the vacuum surge is smoother. In another example, a closed loop feedback on IOP pressure allows a processor to adjust the duty cycle according to a minimally required level of vacuum surge suppression (e.g., a level that the user finds to maintain an acceptable level of followability).

In an example, a system is provided that comprises a graphical user interface (GUI) configured to receive user selection of a followability level. A processor of the system is configured to calculate followability accordingly, as required by AVS the driving waveform. During phacoemulsification, the processor receives pressure sensor readings, and if the processor estimates that an occlusion break or vacuum surge occurs, the processor activates the AVS mechanism to suppress the vacuum surge, while maintaining an acceptable level of followability.

The processor may be further configured to present a recommended followability on the GUI, for user selection, data accumulated during other that is based on phacoemulsification procedures performed by the same user.

The processor can be configured to train a machine learning (ML) algorithm to infer and a recommend followability level for the user to select, wherein the processor is configured to train the ML algorithm using a database of changes to the selected followability made by that user during past phacoemulsification procedures.

Finally, in some cases, the disclosed technique may be applied in conjunction with an operation of an aspiration pump. For example, the AVS may smoothly suppress a vacuum surge by being modulated as described above, giving the aspiration pump time to reverse its operation to reduce the vacuum level in an aspiration line. In this way the physician may have a more continuous mode of work even under occurrences of substantial aspiration occlusions.

### Apparatus Description

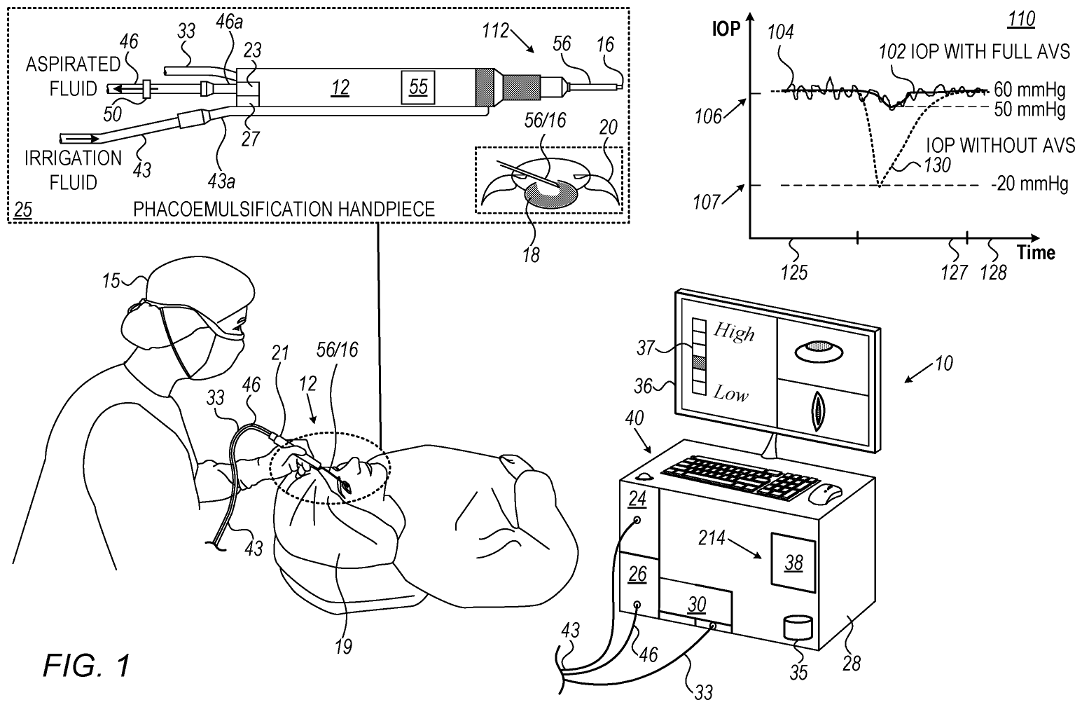

FIG. 1 is a schematic, pictorial view, along with an orthographic side view, of a phacoemulsification apparatus 10, in accordance with an example of the present disclosure.

As seen in the pictorial view of phacoemulsification apparatus 10, and in the schematic side view inset 25, a phacoemulsification probe 12 (e.g., a handpiece) comprises a distal end 112 comprising a needle 16 and a coaxial irrigation sleeve 56 that at least partially surrounds needle 16 and creates a fluid pathway between the external wall of the needle and the internal wall of the irrigation sleeve, where needle 16 is hollow to provide an aspiration channel. Moreover, the irrigation sleeve may have one or more side ports at or near the distal end to allow irrigation fluid to flow toward the distal end of the handpiece through the fluid pathway and out of the port(s).

Needle 16 is configured for insertion into a lens capsule 18 of an eye 20 of a patient 19 by a physician 15 to remove a cataract. While needle 16 (and irrigation sleeve 56) are shown in inset 25 as a straight object, any suitable needle may be used with phacoemulsification probe 12, for example, a curved or bent tip needle commercially available from Johnson & Johnson Surgical Vision, Inc., Irvine, CA, USA.

In the shown example, during the phacoemulsification procedure, an irrigation pump 24, comprised in a console 28, pumps irrigation fluid from an irrigation reservoir (not shown) to irrigation sleeve 56 to irrigate the eye. The fluid is pumped via an irrigation tubing line 43 running from console 28 to an irrigation channel 43a of probe 12. In another example, pump 24 may be coupled with, or replaced by, a gravity-fed irrigation source such as a balanced salt solution bottle/bag.

Fluid and waste matter (e.g., emulsified parts of the cataract) are aspirated via hollow needle 16 to a collection receptacle (not shown) by a processor-controlled aspiration pump 26, also comprised in console 28, using aspiration tubing line 46 running from aspiration channel 46a of probe 12 to console 28.

In the shown example, probe 12 includes an irrigation sensor 27 (e.g., pressure sensor) coupled with irrigation channel 43a and an aspiration sensor 23 (e.g., vacuum sensor) coupled with an aspiration channel 46a. Irrigation sensor 27 may be positioned anywhere along tubing line 43 or channel 43a. Likewise, aspiration sensor 23 may be positioned anywhere along tubing line 46 or channel 46a.

Channels 43a and 46a are coupled respectively with irrigation line 43 and aspiration line 46. Pumps 24 and 26 may be any pump known in the art (e.g., peristaltic pump, progressive cavity pump). Using sensors (e.g., as indicated by sensors 27 and/or 23), a processor 38 controls a flow rate of irrigation pump 24 and/or aspiration pump 26 to maintain IOP within prespecified limits. In some cases, typically to protect against a vacuum surge hazard, the system activates AVS module 50 (seen in inset 25) to disconnect aspiration channel 46a from line 46 and aspiration pump 26. AVS module 50 can be autonomous, with a processor inside AVS 50 that receives and processes sensor readings and commands activation of module 50, or module 50 can be commanded from processor 38.

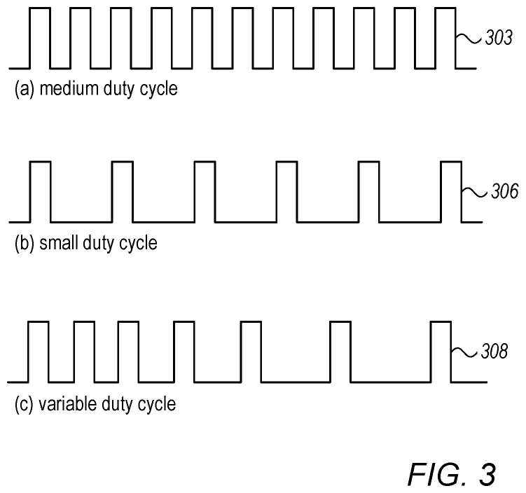

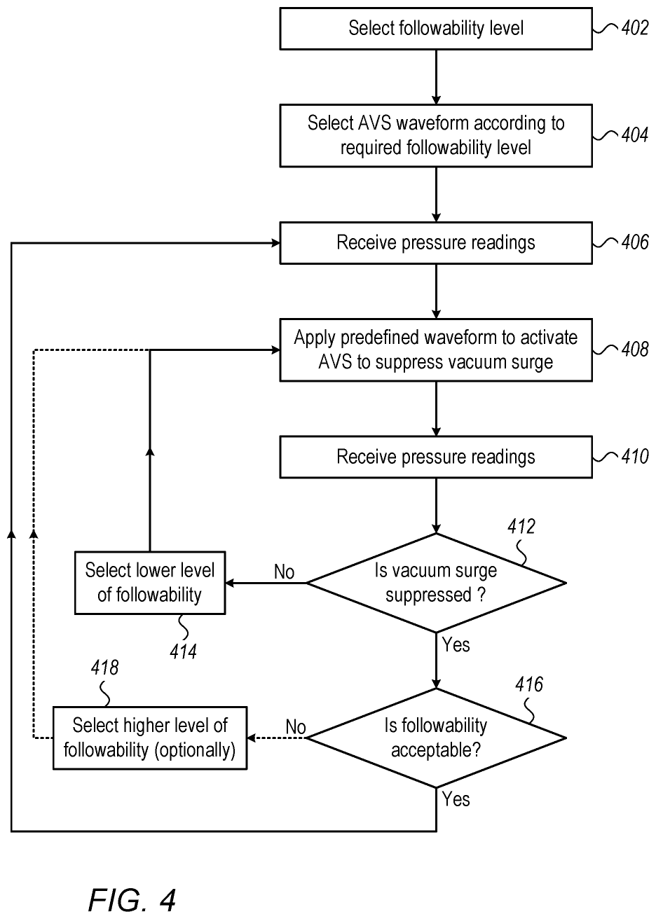

To overcome or minimize the effects of a vacuum surge while maintaining acceptable followability, system 10 allows physician 15 to select a followability level. The processor activates AVS module 50 with a waveform predefined according to the selected followability, as described in FIGS. 3 and 4 below.

In the example of FIG. 1, user interface 40 and display 36 may be integrated into a touch screen graphical user interface (GUI). Display 36 shows a followability level scale 37 that physician 15 can use to select a given discrete level.

For each user there exists some preferable followability level. In the example of FIG. 1, display 36 shows a discrete followability level scale 37. The physician may therefore adjust the followability level, as needed, based on experience. For example, a physician who wishes less disrupted suction power occurring by any possible false positive AVS activation events may tolerate less AVS protection, by selecting a higher followability level on the GUI.

In other examples, the user may adjust the followability level with a virtual slide ruler, enter a numerical value, or make a verbal instruction.

As noted above, processor 38 may control the flow rate of irrigation pump 24 and/or aspiration pump 26, where one of the software modules running in processor 38 is a proportional-integral-derivative (PID) controller 214.

Processor 38 estimates the IOP using readings from the irrigation pressure sensor 27 and an optional empirical offset (if the irrigation pressure is measured at the proximal end of handpiece 12). Readings of sensor 23 give the vacuum level (also called sub-pressure) inside the aspiration channel.

Sensors 27 and 23 may be any sensor known in the art, including, but not limited to, a vacuum sensor or flow sensor. The sensor measurements (e.g., of pressure, vacuum, and/or flow) may optionally be taken close to the distal end of the handpiece where the irrigation outlet and the aspiration inlet are located, so as to provide processor 38 with an accurate indication of the actual measurements occurring within an eye, thus providing a short response time to a control loop comprised in processor 38.

In an example, the same pressure sensor model is used to measure irrigation pressure and aspiration sub-pressure, using different sensor settings/calibrations.

As further shown, phacoemulsification probe 12 includes a piezoelectric crystal 55, coupled to a horn (not shown), that drives needle 16 to vibrate in a resonant vibration mode that is used to break a cataract into small pieces during a phacoemulsification procedure. Console 28 comprises a piezoelectric drive module 30, coupled with the piezoelectric crystal, using electrical wiring running in cable 33.

Processor 38 may receive user-based commands via a user interface 40, which may include setting a vibration mode and/or frequency of the piezoelectric crystal, and setting or adjusting an irrigation and/or aspiration rate of the irrigation pump 24 and aspiration pump 26. Optionally, user interface 40 includes a foot pedal. Processor 38 may receive user-based commands via a user interface 40, which may include needle 16 stroke amplitude settings and initiation of irrigation and/or aspiration. In an example, the physician uses a foot pedal (not shown) as a means of control. For example, a foot pedal may have a treadle that is moveable in a pitch direction and the available pitch travel may be divided into multiple functionality zones or positions. Foot pedal position one activates only irrigation, foot pedal position two activates both irrigation and aspiration, and foot pedal position three adds needle 16 vibration. Additionally, or alternatively, processor 38 may receive user-based commands from controls located in a handle 21 of probe 12.

Some or all of the functions of processor 38 may be combined in a single physical component or, alternatively, implemented using multiple physical components. These physical components may comprise hard-wired or programmable devices, or a combination of the two. In some examples, at least some of the functions of processor 38 may be carried out by suitable software stored in a memory 35 (as shown in FIG. 1). This software may be downloaded to a device in electronic form, over a network, for example. Alternatively, or additionally, the software may be stored in tangible, non-transitory computer-readable storage media, such as optical, magnetic, or electronic memory.

The apparatus shown in FIG. 1 is simplified for clarity of presentation. For example, the disclosed AVS scheme may be applied using alternative or additional control devices of the system, such as other valves (e.g., a bypass valve).

### Full Suppression of Vacuum Surge

Reference is now made to Graph 110 of FIG. 1. Physician 15 typically works (e.g., during time durations 125 and 128) at a nominal TOP level 106 (e.g., 60 mmHg). Due to an occlusion break during phacoemulsification, IOP may abruptly fall (130) within a time duration 127 to a hazardous low level IOP 107 (e.g., −20 mmHg).

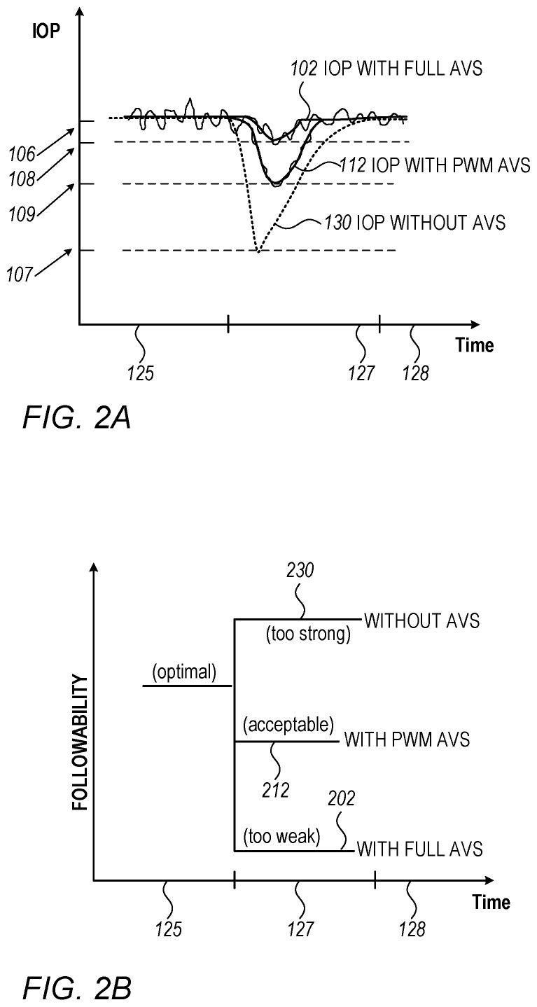

At some point the AVS module 50 is activated to reverse the IOP dip before it reaches 107. Curve 102 is an idealized smooth variation in IOP when AVS module 50 cuts off aspiration to avoid vacuum surge 130. (An actual curve 104 demonstrates how IOP fluctuates about level 102). As seen, cutting off aspiration is very efficient, with IOP drop suppressed almost entirely (IOP momentarily drops to 50 mmHg). However, as described in FIG. 2B below, this drastic response of the AVS modules also overly suppresses followability.

### Modulated Suppression of Vacuum Surge

FIGS. 2A and 2B are graphs that schematically show different levels of suppression of a vacuum surge with resulting respective followability levels, in accordance with examples of the present disclosure. FIG. 2A shows three curves. Curve 102 is the aforementioned IOP profile when an AVS module is applied fully to suppress a vacuum surge. A resulting respective followability profile 202 is given in FIG. 2B. As seen, full activation of AVS module 50 results in practical loss of followability.

Not operating AVS 50 module at all results in vacuum surge curve 130 which is also accompanied by overly strong suction, as described in FIG. 2B by followability curve 230.

The inventors observed that, between the two undesired profiles of vacuum surge respectively undesired followability, exists a range of acceptable vacuum surge levels and acceptable respective followability levels, as schematically described by curves 112 and 212. To access that useful range, the disclosed technique may use pulse width modulation (PWM) to activate AVS module 50 in a measured manner as described in FIG. 3 below.

### Activating AVS with Improved Followability

FIG. 3 is a schematic, pictorial illustration of different waveforms applied to AVS module 50 to suppress a vacuum surge, in accordance with examples of the present disclosure. Waveforms 303 and 306 correspond respectively to lower and higher levels of required followability. The medium duty cycle waveform 303 generates stronger anti-vacuum response than waveform 306, by maintaining the valve closed for long duration over each period. Correspondingly, using 303 results in lower followability (e.g., lower effective suction power). The processor is configured to activate the AVS in a predefined pulsed manner (e.g., predefined waveform such as 303 or 306 with a given duty cycle) according to the selected followability level.

In FIG. 1, display 36 shows a followability level scale 37 that physician 15 can use to select a given discrete level of followability. The processor correspondingly selects a driving waveform. For example, the processor selects to reduce the duty cycle of AVS activation (e.g., waveform 306, compared to waveform 303) since the physician has selected an increased selected level of followability.

In another example, the processor selects from waveforms designed with increasing intervals (e.g., decreasing duty cycles), such as chirp waveform 308 such that addressing the vacuum surge will be smoother and the accompanying vacuum surge less pronounced.

### Method of Activating AVS with Improved Followability

FIG. 4 is a flow chart schematically illustrating a method to suppress vacuum surge while maintaining followability, in accordance with an example of the present disclosure. The algorithm, according to the presented example, carries out a process that begins with physician 15 selecting a followability level (e.g., from scale 37) or accepting a system selection, at a followability selecting step 402.

Next, at a calculation step 404, the processor calculates, or selects a predefined, AVS-driving waveform, according to the required followability to be used for suppressing a vacuum surge. The calculation can be based on a monotonically decreasing relation between followability and waveform duty cycle that is obtained experimentally (e.g., a linear relation with the coefficient that is determined empirically).

Next, during phacoemulsification, processor 38 receives pressure readings, at pressure readings receiving step 406. When the processor identifies a vacuum surge or occlusion break, the processor applies the AVS-driving waveform of step 404 to activate the AVS mechanism to suppress the vacuum surge, at a vacuum surge suppression step 408.

During vacuum surge suppression, processor 38 receives pressure readings, at pressure readings receiving step 410.

At a checking step 412, the processor checks and estimates, from pressure readings, if the vacuum surge is sufficiently suppressed.

If the processor concludes that a vacuum surge is insufficiently suppressed, the processor selects a lower followability and a respective waveform (e.g., with a higher duty cycle), at AVS parameter selection step 414. The process then returns to step 408.

If the processor concludes that a vacuum surge is sufficiently suppressed, the processor checks if the followability is sufficient (e.g., by checking if user followability level entered (e.g., command) in the GUI changed), at followability level checking step 416. If the processor finds the followability acceptable, the process returns to step 406, to receive new sensor readings.

Optionally, if processor 38 finds a requirement to increase followability, the processor selects a higher followability level in followability selection step 418. The process then returns to step 408.

If, at step 418, the processor finds no new requirement on followability, the AVS activation and the processor continue to monitor pressure readings as long as the phacoemulsification procedure continues, by returning to step 406.

Optionally, at the end of the phacoemulsification procedure, processor 38 may suggest an updated followability based on accumulated data on vacuum surge performance under selected followability from one or more procedures performed by the physician. In some examples, processor 38 receives input from the physician or clinical professional, and, based on this input, processor 38 may compute an adjusted followability. Optionally, a machine learning algorithm may be applied to adapt a suggested followability to the skill and style of the physician.

### EXAMPLES

### Example 1

A system (10) includes an anti-vacuum surge (AVS) mechanism (50) and a processor (38). The AVS mechanism includes a processor-controlled valve coupled with an aspiration line (46) or on an aspiration channel (46a) of the system, the AVS mechanism configured to suppress a vacuum surge during the phacoemulsification procedure by closing the processor-controlled valve. The processor (38) is configured to (i) during the phacoemulsification procedure, receive pressure readings from one or more sensors (23, 27), (ii) using the pressure readings, estimate an occurrence of a vacuum surge, and (iii) upon estimating the occurrence of the vacuum surge, activate the AVS mechanism (50) in a predefined pulsed manner (303, 306, 308) to respectively activate the valve to suppress the vacuum surge.

### Example 2

The system (10) according to claim 1, wherein the processor (38) is configured to activate the AVS mechanism (50) in a predefined pulsed manner by monotonically increasing (308) intervals between pulses that activate the AVS mechanism.

### Example 3

The system (10) according to any of claims 1 and 2, and further comprising a user interface (36, 37, 40), configured to receive a user-selected level of followability wherein activation of the AVS mechanism (50) in the predefined pulsed manner to suppress the vacuum surge comprises maintaining the selected level (37) of followability.

### Example 4

The system (10) according to any of claims 1 through 3, wherein the processor (38) is configured to activate the AVS mechanism (50) in a predefined pulsed manner by reducing a duty cycle of the AVS mechanism activation with increased selected level (37) of followability.

### Example 5

The system (10) according to any of claims 1 through 3, wherein the processor (38) is configured to adjust the user-selected followability during the phacoemulsification procedure based on AVS mechanism (50) activation data accumulated during the phacoemulsification procedure.

### Example 6

The system (10) according to any of claims 1 through 3, wherein the processor (38) is further configured to, based on accumulated data during other phacoemulsification procedures performed by a same user, present (36, 37) to the user a user-specific recommended followability for the user to select (36, 37, 40).

### Example 7

The system (10) according to claim 3, wherein the user interface (36, 37, 40) is configured to receive the user-selected followability by one selected from the group consisting of the user adjusting a virtual slide ruler, the user selecting (37) a level out of a set of discrete levels, the user entering (40) a numerical value of the user-selected followability, and receiving a verbal instruction made by the user.

### Example 8

The system (10) according to any of claims 1 through 3, wherein the processor (38) is further configured to train a machine learning (ML) algorithm to infer and recommend a user-specific followability for the user to select, wherein the processor is configured to train the ML algorithm using a plurality of recorded changes to the selected followability that the same user made during past phacoemulsification procedures.

### Example 9

A method includes providing an anti-vacuum surge (AVS) mechanism (50) comprising a processor-controlled valve configured to be coupled with an aspiration line (46) or an aspiration channel (46a) of a phacoemulsification system, wherein the AVS mechanism is configured to suppress a vacuum surge during a phacoemulsification procedure by closing the processor-controlled valve. Pressure readings are received during the phacoemulsification procedure, from one or more sensors (23, 27). Using the pressure readings, an occurrence of a vacuum surge is estimated. Upon estimating the occurrence of the vacuum surge, the AVS mechanism is activated in a predefined pulsed manner (303, 306, 308) to respectively activate the processor-controlled valve to suppress the vacuum surge.

It will be appreciated that the examples described above are cited by way of example, and that the present disclosure is not limited to what has been particularly shown and described hereinabove. Rather, the scope of the present disclosure includes both combinations and sub-combinations of the various features described hereinabove, as well as variations and modifications thereof which would occur to persons skilled in the art upon reading the foregoing description and which are not disclosed in the prior art. Documents incorporated by reference in the present patent application are to be considered an integral part of the application except that to the extent any terms are defined in these incorporated documents in a manner that conflicts with the definitions made explicitly or implicitly in the present specification, only the definitions in the present specification should be considered.

1. A system, comprising:
an anti-vacuum surge (AVS) mechanism comprising a processor-controlled valve coupled with an aspiration channel of the system, the AVS mechanism configured to suppress a vacuum surge during the phacoemulsification procedure by closing the processor-controlled valve; and
a processor, which is configured to:
during the phacoemulsification procedure, receive pressure readings from one or more sensors;
using the pressure readings, estimate an occurrence of a vacuum surge; and
upon estimating the occurrence of the vacuum surge, activate the AVS mechanism in a predefined pulsed manner to respectively activate the valve to suppress the vacuum surge.

2. The system according to claim 1, wherein the processor is configured to activate the AVS mechanism in a predefined pulsed manner by monotonically increasing intervals between pulses that activate the AVS mechanism.

3. The system according to claim 1, further comprising:
a user interface, configured to receive a user-selected level of followability wherein activation of the AVS mechanism in the predefined pulsed manner to suppress the vacuum surge comprises maintaining the selected level of followability.

4. The system according to claim 3, wherein the processor is configured to activate the AVS mechanism in a predefined pulsed manner by reducing a duty cycle of the AVS mechanism activation with increased selected level of followability.

5. The system according to claim 3, wherein the processor is configured to adjust the user-selected followability during the phacoemulsification procedure based on AVS mechanism activation data accumulated during the phacoemulsification procedure.

6. The system according to claim 3, wherein the processor is further configured to, based on accumulated data during other phacoemulsification procedures performed by a same user, present to the user a user-specific recommended followability for the user to select.

7. The system according to claim 3, wherein the user interface is configured to receive the user-selected followability by one selected from the group consisting of the user adjusting a virtual slide ruler, the user selecting a level out of a set of discrete levels, the user entering a numerical value of the user-selected followability, and receiving a verbal instruction made by the user.

8. The system according to claim 3, wherein the processor is further configured to train a machine learning (ML) algorithm to infer and recommend a user-specific followability for the user to select, wherein the processor is configured to train the ML algorithm using a plurality of recorded changes to the selected followability that the same user made during past phacoemulsification procedures.

9. A method, comprising:
providing an anti-vacuum surge (AVS) mechanism comprising a processor-controlled valve configured to be coupled with an aspiration channel of a phacoemulsification system, wherein the AVS mechanism is configured to suppress a vacuum surge during a phacoemulsification procedure by closing the processor-controlled valve;
during the phacoemulsification procedure, receiving pressure readings from one or more sensors;
using the pressure readings, estimating an occurrence of a vacuum surge; and
upon estimating the occurrence of the vacuum surge, activating the AVS mechanism in a predefined pulsed manner to respectively activate the processor-controlled valve to suppress the vacuum surge.

10. The method according to claim 9, wherein activating the AVS mechanism in a predefined pulsed manner comprises monotonically increasing intervals between pulses that activate the AVS mechanism.

11. The method according to claim 9, further comprising:
in a user interface, receiving a user-selected level of followability and maintaining the user-selected level of followability during activation of the AVS mechanism.

12. The method according to claim 11, wherein activating the AVS mechanism in a predefined pulsed manner comprises reducing a duty cycle of the AVS mechanism activation with increased selected level of followability.

13. The method according to claim 11, wherein adjusting the user-selected followability during the phacoemulsification procedure comprises adjusting based on AVS mechanism activation data accumulated during the phacoemulsification procedure.

14. The method according to claim 11, further comprising, based on accumulated data during other phacoemulsification procedures performed by a same user, presenting to the user a user-specific recommended followability for the user to select.

15. The method according to claim 11, wherein receiving the user-selected followability comprises one selected from the group consisting of the user adjusting a virtual slide ruler, the user selecting a level out of a set of discrete levels, the user entering a numerical value of the user-selected followability, and receiving a verbal instruction made by the user.

16. The method of claim 11, further comprising training a machine learning (ML) algorithm to infer and recommend a user-specific followability for the user to select, wherein training the ML algorithm comprises using a plurality of recorded changes to the selected followability that the same user made during past phacoemulsification procedures.
